(19) United States
(12) Patent Application Publication
(10) Pub. No.: US 2026/0053507 A1
(43) Pub. Date: Feb. 26, 2026

(54) SURGICAL GOUGE ASSEMBLY

(71) Applicants: Northwestern University, Evanston, IL (US); The Texas A & M University System, College Station, TX (US); Ann & Robert H. Lurie Children's Hospital of Chicago, Chicago, IL (US)

(72) Inventors: Nikhil Dipak Shah, Yorba Linda, CA (US); Narainsai K. Reddy, Bellaire, TX (US); Akira Yamada, Chicago, IL (US); Basil Alias, The Colony, TX (US)

(21) Appl. No.: 19/102,740

(22) PCT Filed: Aug. 22, 2023
(86) PCT No.: PCT/US2023/072610

§ 371 Date: Feb. 10, 2025

Related U.S. Application Data
(60) Provisional application No. 63/400,548, filed on Aug. 24, 2022.

(51) Int. Cl. A61B 17/16

(52) U.S. Cl. CPC A61B 17/1613; A61B 17/1604

(57) ABSTRACT

A handle assembly for a gouge includes a handle, where a distal end of the handle includes first threads. The handle assembly includes a grip that has an interior channel. At least a portion of the interior channel includes second threads that mate with the first threads on the distal end of the handle to secure the grip to the handle. The handle assembly also includes a collet. A distal end of the collet includes an opening that is sized to receive a proximal end of a gouge. The handle assembly further includes a collar that is sized to receive the collet, and that mounts onto a distal end of the grip.

### CROSS-REFERENCE TO RELATED APPLICATION

The present application claims the priority benefit of U.S. Provisional Patent App. No. 63/400,548 filed on Aug. 24, 2022, the entire disclosure of which is incorporated by reference herein.

### BACKGROUND

Reconstructive surgery often requires precise manipulation of autologous cartilage to achieve an appropriate form. This is especially true in auricle reconstruction for patients with trauma or congenital microtia. In order to achieve an appropriate contour of the helix, antihelix, and other structural components, surgical gouges are routinely used. The aesthetic and surgical outcome of such reconstruction procedures is heavily influenced by the manufacturing quality, alloy selection, and post-processing of the specific surgical gouge utilized during the operation. Multiple different gouge styles and alloys from different healthcare companies have been utilized in such reconstructive surgeries.

### SUMMARY

An illustrative handle assembly for a gouge includes a handle. A distal end of the handle includes first threads. The assembly also includes a grip that has an interior channel, where at least a portion of the interior channel includes second threads that mate with the first threads on the distal end of the handle to secure the grip to the handle. The assembly includes a collet, where a distal end of the collet includes an opening that is sized to receive a proximal end of a gouge. The assembly also includes a collar that is sized to receive the collet, where the collar mounts onto a distal end of the grip.

In an illustrative embodiment, the grip includes a distal portion that has third threads, the collar includes fourth threads internal to the collar, and the fourth threads mate with the third threads to secure the collar to the grip. In another embodiment, the collet includes a pair of slots that extend to the opening in the collet, and the pair of slots compress the opening in response to the fourth threads mating with the third threads to secure the collar to the grip. In one embodiment, the pair of slots extend from the opening in the collet to a central portion of the collet. The pair of slots are also configured to receive at least a portion of the gouge. In another illustrative embodiment, compression of the opening secures the gouge within the opening at a desired cutting depth and a desired angle.

In another embodiment, the grip includes a sleeve positioned in between the distal end of the grip and a proximal end of the grip. In another embodiment, the interior channel of the grip runs an entire length of the grip. In one embodiment, the interior channel includes a distal channel portion and a proximal channel portion, and the distal channel portion tapers down in size to connect to the proximal channel portion. In another embodiment, the collar includes an opening that aligns with the opening in the collet when the collet is positioned within the collar.

An illustrative method of forming components of a handle assembly for a gouge includes forming a handle with a proximal end and a distal end, where the distal end of the handle is formed to include first threads. The method also includes forming a grip that has an interior channel, where at least a portion of the interior channel includes second threads that mate with the first threads on the distal end of the handle to secure the grip to the handle. The method also includes forming a collet such that a distal end of the collet includes an opening that is sized to receive a proximal end of a gouge. The method further includes forming a collar that is sized to receive the collet, where the collar mounts onto a distal end of the grip

In another embodiment, the method includes mounting the grip to the handle by mating the first threads with the second threads, positioning the collet within the collar, and mounting the collar to the distal end of the grip. The method can also include forming the grip to include a distal portion that has third threads, where the collar is formed to include fourth threads internal to the collar, and where the fourth threads mate with the third threads to secure the collar to the grip. The method can also include forming the collet to include a pair of slots that extend to the opening in the collet, where the pair of slots compress the opening in response to the fourth threads mating with the third threads to secure the collar to the grip. In one embodiment, the pair of slots extend from the opening in the collet to a central portion of the collet. In another embodiment, the collet is formed to include two pair of slots that extend to the opening in the collet. In one embodiment, the pair of slots is configured to receive at least a portion of the gouge. In another embodiment, the interior channel of the grip runs an entire length of the grip. In one embodiment, the interior channel includes a distal channel portion and a proximal channel portion, and where the distal channel portion tapers down in size to connect to the proximal channel portion. In another embodiment, the collar is formed to include an opening that aligns with the opening in the collet when the collet is positioned within the collar.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1A is a perspective view of a handle assembly for a gouge in accordance with an illustrative embodiment.

FIG. 1B is a side view of the handle assembly and includes a section line A-A in accordance with an illustrative embodiment.

FIG. 1C is a cross-sectional view of the handle assembly along the section line A-A in accordance with an illustrative embodiment.

FIG. 1D is an exploded view of the handle assembly in accordance with an illustrative embodiment.

FIG. 1E is a perspective view of the handle assembly with a gouge mounted therein in accordance with an illustrative embodiment.

FIG. 1F is a side view of the handle assembly with the gouge mounted therein in accordance with an illustrative embodiment.

FIG. 1G is a cross-sectional view of the handle assembly taken along the section line A-A of FIG. 1F in accordance with an illustrative embodiment.

FIG. 2A is a side view of a handle of the handle assembly in accordance with an illustrative embodiment.

FIG. 2B is a perspective view of the handle of the handle assembly in accordance with an illustrative embodiment.

FIG. 2C is an end view of a proximal end of the handle in accordance with an illustrative embodiment.

FIG. 2D is an end view of a distal end of the handle in accordance with an illustrative embodiment.

FIG. 3A is a perspective view of a grip of the handle assembly in accordance with an illustrative embodiment.

FIG. 3B is a side view of the grip of the handle assembly in accordance with an illustrative embodiment.

FIG. 3C is an end view of a distal end of the grip and includes a section line D-D in accordance with an illustrative embodiment.

FIG. 3D is a cross-sectional view of the grip along the section line D-D in accordance with an illustrative embodiment.

FIG. 4A is a perspective view of a collar of the handle assembly in accordance with an illustrative embodiment.

FIG. 4B is a side view of the collar of the handle assembly in accordance with an illustrative embodiment.

FIG. 4C is an end view of a proximal end of the collar in accordance with an illustrative embodiment.

FIG. 4D is an end view of a distal end of the collar in accordance with an illustrative embodiment.

FIG. 5A is a perspective view of a collet (or inner clamp) that secures a gouge within the handle assembly in accordance with an illustrative embodiment.

FIG. 5B is a side view of the collet in accordance with an illustrative embodiment.

FIG. 5C is an end view of a distal end of the collet in accordance with an illustrative embodiment.

### DETAILED DESCRIPTION

Commercially available surgical gouges typically utilize stainless steel for the gouges, along with rudimentary aluminum or stainless steel handles that include a set screw to hold the gouge in place. There are currently no handles on the market that are capable of quickly setting disposable or reusable gauges into a handle, allowing for multiple degrees of movement and adjustment of the length of the gouge. Additionally, many of the currently available handles are not compatible with the sterilization process required at major hospitals as per Food and Drug Administration (FDA) guidelines in the United States, which means that they cannot be cleaned and sterilized for reuse. Traditional metal-made handles are heavy, not ergonomically designed, and lacking in multiple features that are addressed by the assembly described herein.

More specifically, issues with currently available gouges include difficulty exchanging and reusing blades both during and after surgery, as new blades must be used for each surgery because sterilization causes certain blades to oxidize. Heaviness of the handle makes it difficult to maneuver and increases surgeon fatigue, which can rapidly aggravate hand tremor. Lack of ergonomics of the handle also prevents easy manipulation during surgery. Additionally, achieving appropriate cartilage form involves manipulation of the gouge to various angles and constant extension and retraction of the blade, which is not readily feasible in traditional gouges. Traditional gouges also do not allow for use of a pencil grip, which is the ideal ergonomic position for efficient use.

Given the lack of an ergonomic, comfortable, and adjustable handle that can be sterilized, many surgeons performing microtia and other reconstructive surgeries do not use gouges, as it is thought that the gouges currently available on the market can lead to substandard surgical outcomes. As an alternative, these surgeons often use skin biopsy punches to perform such procedures. However, while the biopsy punches can lead to better aesthetic outcomes as compared to currently available gouges, they are not ergonomically and surgically efficient to use.

Described herein is a surgical gouge assembly that couples an ergonomic handle design with the gouge to enable quick table side adjustments, while also being compatible with sterilization, cleanability, and biocompatibility guidelines from the FDA. The proposed gouge system will enable surgeons, scrub nurses, and surgical technologists to utilize gouge blades with efficiency in surgery, while also being compatible with sterilization techniques. In an illustrative embodiment, a handle of the gouge system can be in the form of a multi-piece assembly that can be made from a multitude of different materials to allow for fixation of a wide range of disposable/reusable surgical gouge blades at multiple positions, depths, and angles in order to optimize surgical precision and speed. As one example of the gouge blade type, many surgeons believe that the quality of gouges made from a unique steel alloy manufactured in Japan remains unmatched.

In an illustrative embodiment, the gouge system includes a multi-positional adjustable handle designed to securely hold, position, extend, and retract reusable or disposable surgical gouges. The proposed handle enables the user to adjust the gouge angulation and cutting depth quickly during surgery. The device enables reusable or disposable surgical gouges of variable diameters and length to be inserted. In an illustrative embodiment, the handle can be composed of 3 pieces that are connected to each other through internal and external threads. Alternatively, handle components can be connected by a method other than threads, such as a snap-fit connector, friction fit, etc. The assembly can be made from a number of different materials, and these different materials can be used for unique surgical situations. For example, the proposed system can conserve its physical design across different use cases that require different materials, such as Delrin (Polyoxymethylene) non-porous plastics, radiolucent materials, single use plastics, etc. In addition to plastics, the proposed handle assembly can be made from a wide variety of metals such as stainless steel (303 or 304, 316L), titanium (Ti6Al4V), cobalt alloys (CoCr), aluminum (Al 6061), etc.

The handle and gouge attachment point of the proposed gouge system allow an internal collet to be held in place while also enabling pass through of a reusable or disposable surgical gouge to allow for adjustment of the length of the instrument tip. The collet allows the instrument to constrict the reusable or disposable gouge tip without allowing movement in the distal tip that could potentially translate to excessive removal of in situ or autograft tissue. Also, the tip of the device allows the collet to stay in place and provides an ergonomic position for a surgeon's hand, but also generates the force needed to close the collet. Additionally, the tip allows for quick length adjustments and angulation adjustments that enable the surgeon, first assist, scrub nurse, or surgical technician to adjust the position of the reusable or disposable surgical gouge according to the user's preference.

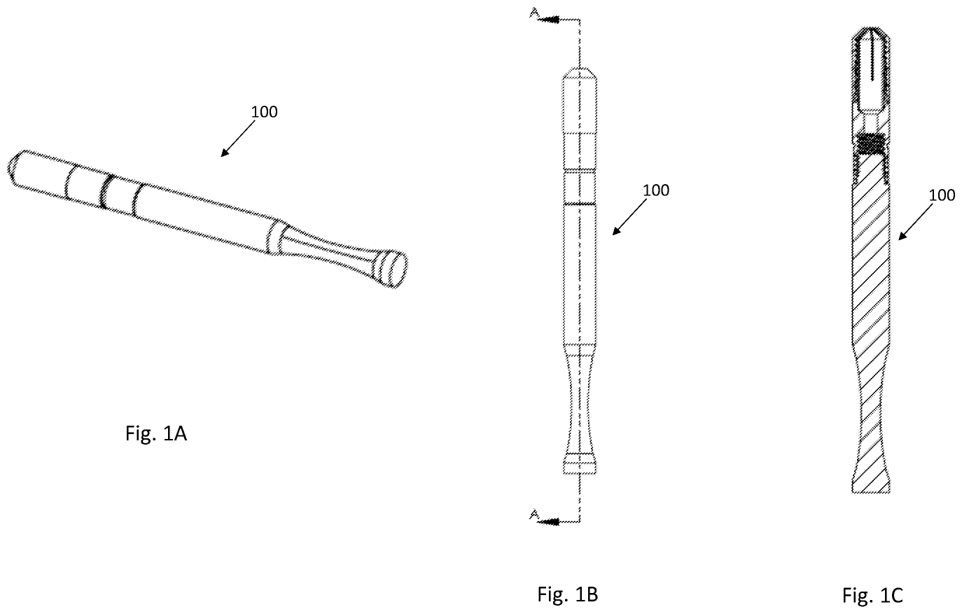

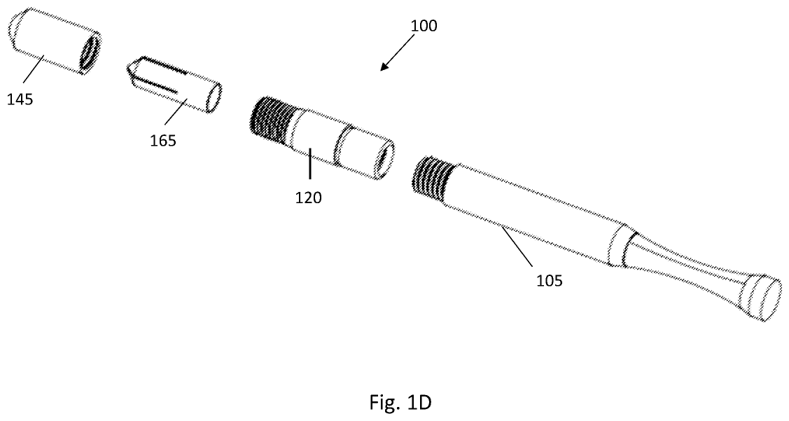

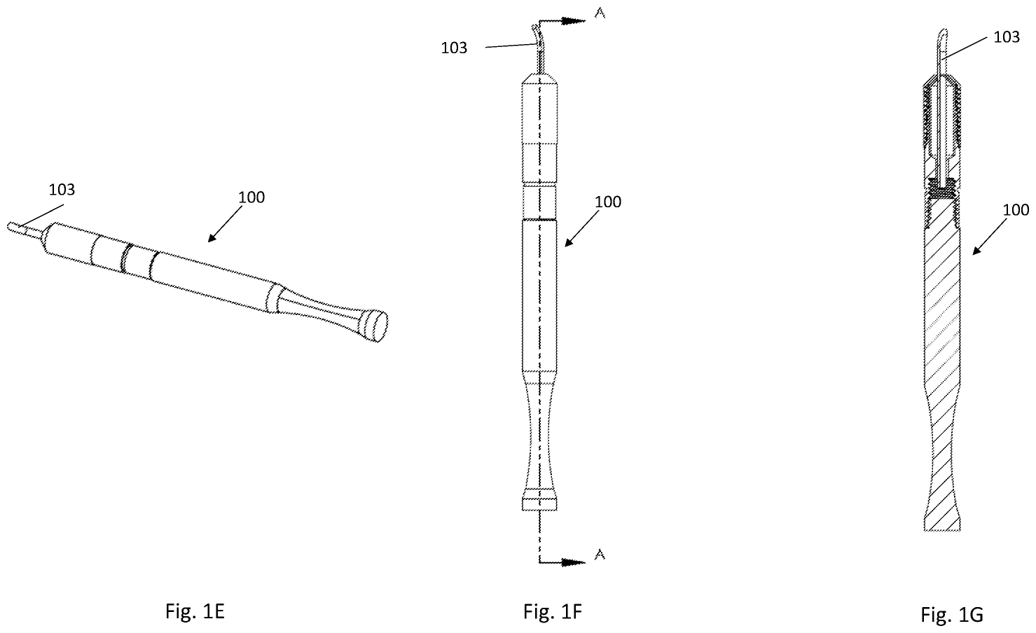

The proposed gouge assembly can be used in numerous different surgical environments. For example, the gouge system can be used for auricle reconstruction, rhinoplasty procedures, notchplasty procedures, orthopedic procedures to contour bone or remove cement, in situ split costal cartilage graft harvesting, harvesting bone or cartilage for grafts, pubectomy in patients undergoing delayed anastomotic urethroplasty for pelvic fracture urethral injury, mastoid gouge for bilateral otoplasty, bone cutting, and various other types of specialty surgeries in the fields of neurosurgery, otolaryngology, orthopedic surgery, urology, plastic surgery, maxillofacial and craniofacial surgery, total hip arthroplasty, laminectomy, impacted wisdom tooth extraction, cochlear implant, mastoidectomy, etc. FIG. 1A is a perspective view of a handle assembly 100 for a gouge in accordance

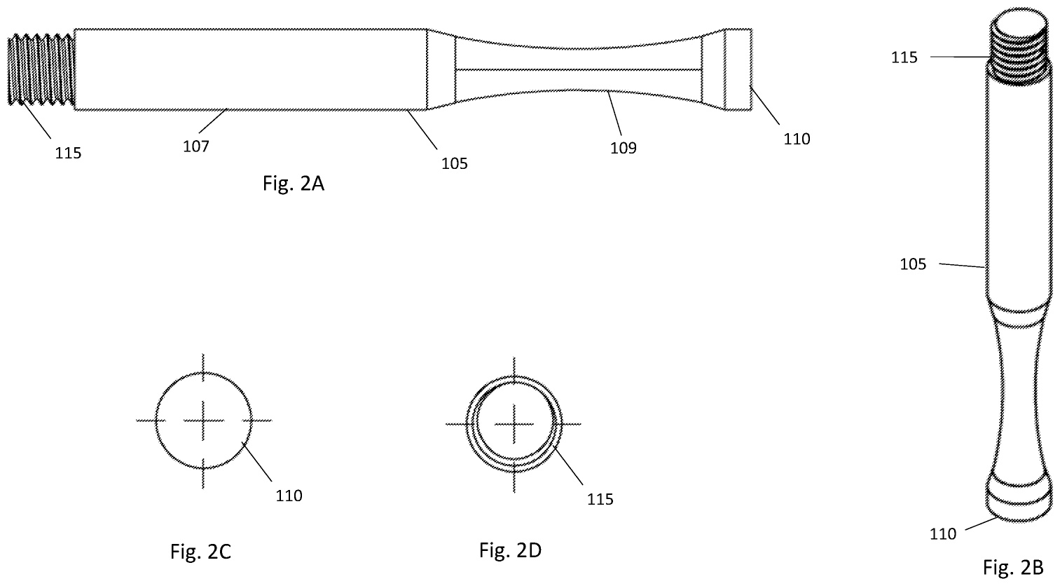

with an illustrative embodiment. FIG. 1B is a side view of the handle assembly 100 and includes a section line A-A in accordance with an illustrative embodiment. FIG. 1C is a cross-sectional view of the handle assembly 100 along the section line A-A in accordance with an illustrative embodiment. FIG. 1D is an exploded view of the handle assembly in accordance with an illustrative embodiment. The exploded view shows elements of the handle assembly 100, including a handle 105, a grip 120, a collet 165, and a collar 145. These elements are described in more detail below with reference to FIGS. 2-5.

FIG. 1E is a perspective view of the handle assembly 100 with a gouge 103 mounted therein in accordance with an illustrative embodiment. The gouge 103 is meant to serve as an illustrative example. In alternative embodiments, gouges having a different shape, diameter, and/or length may be used. FIG. 1F is a side view of the handle assembly 100 with the gouge 103 mounted therein in accordance with an illustrative embodiment. As shown, FIG. 1F includes a section line A-A. FIG. 1G is a cross-sectional view of the handle assembly 100 taken along the section line A-A of FIG. 1F in accordance with an illustrative embodiment.

FIG. 2A is a side view of a handle 105 of the handle assembly in accordance with an illustrative embodiment. FIG. 2B is a perspective view of the handle 105 of the handle assembly in accordance with an illustrative embodiment. FIG. 2C is an end view of a proximal end 110 of the handle 105 in accordance with an illustrative embodiment. FIG. 2D is an end view of a distal end 115 of the handle 105 in accordance with an illustrative embodiment. As used herein, the term proximal refers to a portion of the handle assembly that is furthest from a cutting end of a mounted gouge, and the term distal refers to a portion of the handle assembly that is closest to the cutting end of the mounted gouge. As shown, the distal end 115 of the handle is threaded with male threads to receive a grip portion of the handle assembly, as discussed in more detail below. In an alternative implementation, the distal end 115 of the handle 105 can include a different type of mount (other than threads), such as a snap-fit connection, tongue-and-groove connection, friction fit connection, etc.

As also shown, the handle 105 includes a cylindrical portion 107 adjacent to the distal end 115 and a tapered portion 109 positioned in between the cylindrical portion 107 and the proximal end 110. The tapered portion 109 tapers such that its diameter varies along its length, with a first end of the tapered portion 109 matching a diameter of the cylindrical portion 107 and a second end of the tapered portion 109 matching a diameter of the proximal end 110 of the handle 105. The smaller diameter of the tapered portion 109 in between the cylindrical portion 107 and the proximal end 110 provides a comfortable hand rest for the user. Specifically, when using a pencil grip, the tapered portion 109 is designed to comfortably rest on the hand, in between the user's thumb and index finger as one example.

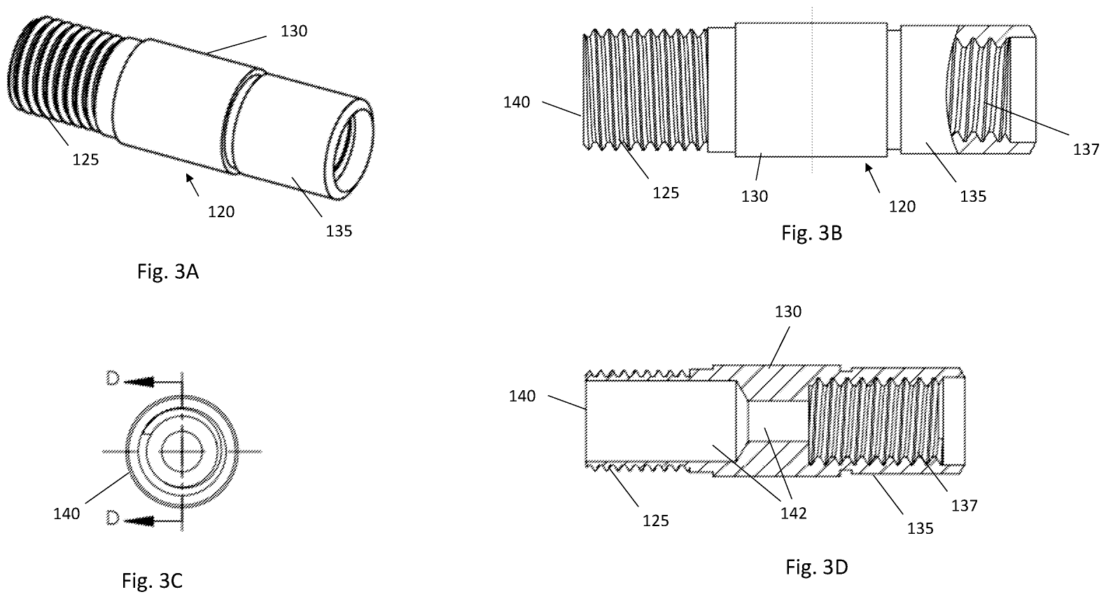

FIG. 3A is a perspective view of a grip 120 of the handle assembly in accordance with an illustrative embodiment. FIG. 3B is a side view of the grip 120 of the handle assembly in accordance with an illustrative embodiment. As shown, the grip 120 includes a distal portion 125 with male threads, a central sleeve 130, and a proximal portion 135 with female threads 137 therein. In an illustrative embodiment, the grip 120 is formed as a monolithic component without moving parts. The side view of FIG. 3B includes a partial cross-sectional view that depicts the female threads 137, which are designed to mate with male threads at the distal end 115 of the handle 105 to secure the handle 105 to the grip 120. In some embodiments, an epoxy, glue, or another adhesive can be used to secure the male threads at the distal end 115 of the handle 105 with the female threads 137 on the interior of the grip 120. FIG. 3C is an end view of a distal end 140 of the grip 120 and includes a section line D-D in accordance with an illustrative embodiment. FIG. 3D is a cross-sectional view of the grip along the section line D-D in accordance with an illustrative embodiment.

As shown, the grip 120 includes an interior channel 142 that runs a length of the grip 120. The interior channel 142 includes a distal channel portion that runs within the distal portion 125 of the grip and a proximal channel portion that runs within the central sleeve 130 and the proximal portion 135 of the grip 120. As shown, the distal channel portion of the interior channel 142 is wider than the proximal channel portion therefore, and the distal channel portion tapers down in size to connect to the proximal channel portion. In one embodiment, the interior channel 142 is aligned with another interior channel formed in the handle 105 to accommodate longer gouges that may extend further into the handle assembly.

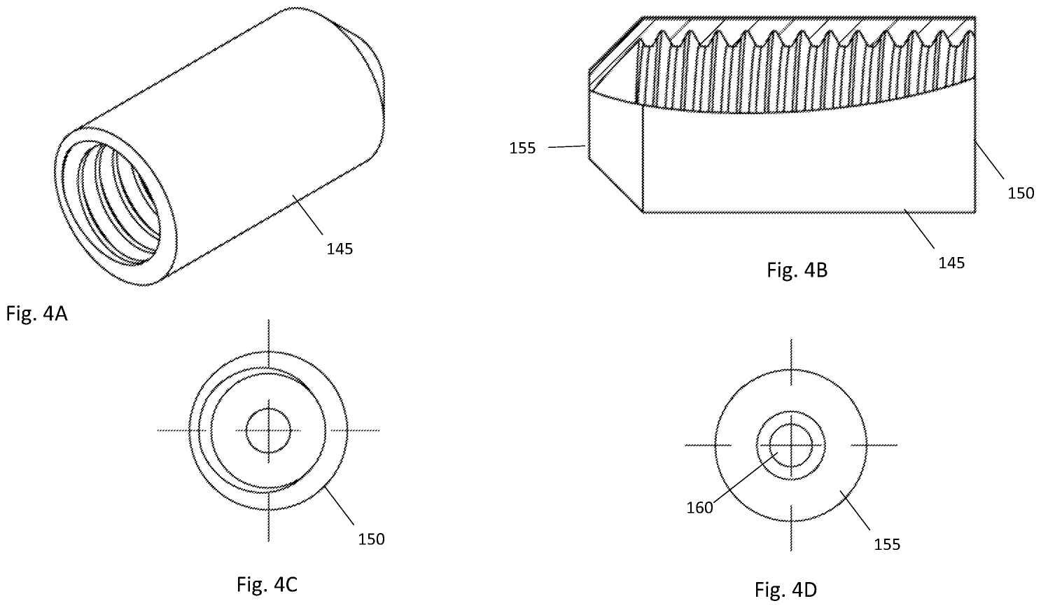

FIG. 4A is a perspective view of a collar (or tip) 145 of the handle assembly in accordance with an illustrative embodiment. FIG. 4B is a side view of the collar 145 of the handle assembly in accordance with an illustrative embodiment. FIG. 4C is an end view of a proximal end 150 of the collar 145 in accordance with an illustrative embodiment. FIG. 4D is an end view of a distal end 155 of the collar 145 in accordance with an illustrative embodiment. An interior of the collar 145 includes threads that mount to male threads on the distal portion 125 of the grip 120. The collar 145 also includes a tapered distal end that includes an opening 160 through which a mounted gouge protrudes.

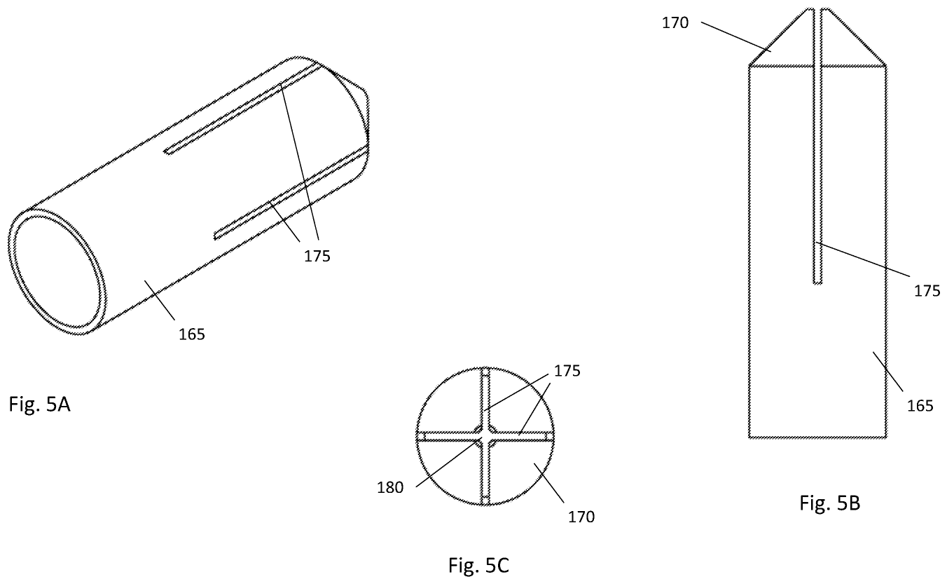

FIG. 5A is a perspective view of a collet (or inner clamp) 165 that secures a gouge within the handle assembly in accordance with an illustrative embodiment. FIG. 5B is a side view of the collet 165 in accordance with an illustrative embodiment. FIG. 5C is an end view of a distal end 170 of the collet 165 in accordance with an illustrative embodiment. As shown, the collet 165 includes slots 175, and the distal end 170 includes an opening 180 through which a distal end (i.e., cutting end) of a mounted gouge protrudes. The slots 175 extend from the opening 180 to a central portion of the collet 165 and allow the collet 165 to compress as the collar 145 is placed over the collet 165 and threaded onto the grip 120. This compression of the collet 165 secures the mounted gouge a desired position/depth. The embodiment shown includes 2 pair of the slots 175. In alternative embodiments, fewer or additional slots may be used.

In practice, the gouge can be mounted within the opening 180 of the collet 165 such that it contacts only the perimeter of the opening 180. A larger gouge may be mounted such that it is in contact with both the perimeter of the opening 180 and a pair of the slots 175 (i.e., a proximal end of the gouge can at least partially rest within the pair of slots 175). The gouge can be mounted by sliding the gouge into the opening 180 and/or a pair of the slots 175. A proximal portion of the collet 165 is received by the distal channel portion of the interior channel 142 of the grip 120, and at least a portion of the collet 165 is also positioned within the collar 145 as the collar is threaded onto the assembly. Specifically, the collar 145 is threaded onto the threads on the distal portion 125 of the grip 120. As the collar 145 is threaded onto the grip 120 (i.e., over the collet 165), the slots 175 of the collet 165 are compressed, which thereby compress the opening 180 in the collet 165 to securely hold the gouge in place at a desired position.

To adjust the cutting depth and/or angle of the gouge, the collar 145 is loosened (i.e., unthreaded from the threads on the distal portion 125 of the grip 120) to decompress the collet 165, and the gouge is either inserted further into the collet 165 (i.e., to shorten the cutting depth) or pulled further out of the collet 165 (i.e., to lengthen the cutting depth). The angle of the gouge can also be adjusted when the collar 145 is loose by rotating the gouge to a desired angle. Once the gouge is at a desired cutting depth and/or angle, the collar 145 is tightened to re-compress the collet 165 and secure the gouge at the new position.

In an illustrative embodiment, the gouges used are curved in shape with a cross-section similar to a crescent moon, and the cross-section is increasingly shallower with every mm increase in size. However, that change in cross section shape does not affect the depth at which the gouge can be inserted into the handle assembly, as the collet 165 has a thin wall thickness and the slots 175 which enable deformation of the distal end of the collet 165. The variations in the size of the gouges are accommodated by the design of collet 165. As discussed, smaller (e.g., 1-2 mm gouges) can be held in place solely by the opening 180 and larger gouges can be held in place by the opening 180 and a pair of the slots 175 across the same plane. In some embodiments, if the gouge is long enough it can be supported by the distal end 115 of the handle 105.

Described below is an example surgical procedure (i.e., total auricular construction) that can be performed more efficiently using the proposed surgical gouge assembly. External ear shape has both aesthetic and functional purpose in human life. Persons born with microtia (small ear or missing ear shape) cannot wear a pair of glasses for reading or vision, cannot wear sunglass for protecting eyes from sunshine, cannot wear a mask to prevent infection from illnesses such as COVID 19, etc.

The current gold standard of care for microtia in children is to create an ear shape from the patient's own rib cartilage, to change it to be similar to natural auricular cartilage, and to cover the transformed cartilage with a local skin envelope. This technique started 60 years ago, but over time the ear framework used has become more realistic and more complex, requiring more sophisticated techniques to construct the architecture of the external ear. To create a natural looking ear construct, the definition of each part of the auricle must be distinct, with natural curves. There are no straight lines in a natural external ear anatomy. As such, the surgeon must make every part of the ear construct with a smooth curve. Once obtained and shaped, the three-dimensional rib cartilage framework is covered with a skin envelope. The minimum thickness of a skin envelope that surgeons can create with good vascular supply to the skin is 2 millimeters (mm) thick. This is thicker than the natural skin of the external ear. Thus, the 3D cartilage framework must be thicker and harder to resist against the force of the skin envelope. As a result, one must create a deeper concave fossa and cavity to accommodate the skin envelope so that the patient has definition in each part of auricular anatomy.

For many surgeons, a specialized gouge is a key surgical instrument to create 3D cartilage framework for total ear reconstruction. The specialized gouge assists with using the patient's own rib cartilage, 6^{th} to 9^{th}, to create cartilage framework for external ear. The cartilage framework is covered with the patient's own skin to make the structure resemble a natural external ear. As noted, the shape of the external ear is complex and the surgeon needs a deep understanding of its three-dimensional architectural structure. For example, it is known that certain dimensions and proportions are required to have the appearance of a normal ear. Characteristics that make a reconstructed ear look like a natural ear include multiple curves, including helix spiral shape, round fossa such as scapha fossa, triangular fossa, and concha cavity.

When one creates a three-dimensional cartilage framework, gouges of different sizes (e.g., 2 mm, 3 mm, 4.4 mm, and 6mm) are often used, along with a number 15 scalpel, and 5-0 stainless steel wires to create the shape of external ear. Five parts of auricle shape components (base frame, helix, antihelix, tragus, concha) are created separately from the 6^{th} to 9^{th} rib cartilage of the patient, and the shape components are assembled by the fine 5-0 stainless wires. To be more precise, an ear framework template of 6 different curves of different sizes is used, and the surgeon tries to match the constructed ear to the normal side of the external ear (if possible). These templates are made based on curve analysis study of a normal external ear.

The five parts of auricle shape components (base frame, helix, antihelix, tragus, and concha) are made using procedures described below. The base frame is made of 6^{th} and 7^{th} rib cartilage of two or three pieces, together with 5-0 stainless wires. Fossa such as scapha fossa and triangular fossa is carved with 2-6 mm gouges down to the bottom of the thickness of the cartilage. Typically, the thickness of the base frame is 5-6 mm in height. Gouges help to create fossa, and curve hollow where the skin envelope will go to make a round surface.

The helix is a spiral curve of the auricle that makes the auricle shape more three dimensional. The helix curve starts at the attachment to the bottom of the base frame (i.e., at crus helicis), and the spiral staircase goes up on to the base framework, creating a round helix curve, along the lateral edge of the base framework. The helix is made of 10-11 cm length 8^{th} rib cartilage. The 8^{th} rib cartilage is cut in half with 5 mm height (if possible), then a medial aspect of the 8^{th} rib is carved with 3 mm or 4.5 mm gouges to make a round surface. The medial aspect is carved with gouges. When helix is attached on top of the base framework, the medial surface is curved as a round curve into one smooth curve where the skin envelope is attached. Again, a slightly angled round gouge makes this work much easier.

The antihelix component is attached on top of the base frame, like the helix component. The antihelix has bifurcation at the top (superior crus and inferior crus), then goes down along the medial edge of the base frame, and goes down to the inter-tragal notch. The antihelix has very mild slope on its lateral side, continuing into scapha fossa. The medial aspect, on the other hand, has almost a vertical wall. Before the antihelix ends at the inter-tragal notch, it has a round prominence, called antitragus. The curve of the antitragus and the tragus creates a U-shape curve as an entrance to the external auditory canal.

Conventional gouges available on the market for rib cartilage carving are straight in shape, and such a shape is not convenient to make curvatures in the scapha fossa, triangular fossa, and concha cavity. A gouge that has a slight curve at the tip of the gouge makes the carving process easier. Two mm and 3 mm width gouges, with slight curvature, are useful to make narrow but deep triangular fossa. Two mm and 3 mm gouges are also useful to create space between the helix and the antitragus. This space is important to avoid a fusion-look between the helix and the antihelix. A 4.5-or 6-mm width gouge works well for making mild slope at scapha fossa between the helix and antihelix.

The tragus is a part of the external ear that creates the shadow to the concha cavity. The physiological purpose of the tragus is to cover “half” of the external auditory canal, so people can hear but at the same time there is protection of the ear canal from foreign bodies or bugs. When one sees the external ear, they see the shadow of the concha cavity, so it is apparent that there is an auditory canal in there. By creating a shadow with the tragus construction, the ear construct looks like it has an external auditory canal, which makes the ear construct more realistic. To create the tragus shape, the inner surface of the tragus must have a concave curve so the skin flap can fit within.

The cap to cymba concha is a small piece of ship shape cartilage that is attached to the back of cymba concha to prevent soft tissue from entering into the cymba concha space. A 2 mm gouge is useful to make a small but deep hollow in this small cap of cartilage. Also, to make more of a distinction to the external auditory canal, a small piece of cartilage is attached to the base frame in the medial surface at the cavum concha. This can be created with either a 4.5 mm or 6 mm gouge. Each of these operations described for the ear reconstruction can be more effectively and efficiently performed using the surgical gouge assembly described herein.

Thus, the gouge assembly described herein allows for widespread use of reusable or disposable unique steel alloy gouges in order to optimize surgical outcomes in a subset of surgeries and medical specialties. These blades allow for easier and more precise manipulation of cartilage and bone. As discussed, there is currently no commercially available surgical handle/blade combination that produces the ideal aesthetic outcomes and functional outcomes that are realized by the proposed gouge system.

Having a lighter, ergonomic handle that fits the ideal gouge, the proposed system improves upon aesthetic, surgical, and functional outcomes given the role of surgical gouges in most cartilage and bone surgeries. Additionally, the variety of materials that can be used in the device enable the device to be used in multiple different settings if needed, as a disposable device, a reusable device, a radiolucent device, etc. Aesthetically, surgeons will achieve more precision using the proposed system. Inferior instruments often result in poorer surgical outcomes, which results in revision surgeries, excess burden on the health care system, and surgeon shopping by patients. Because of this natural tendency of patients, surgeons often compete for patients by advertising their outcomes. Having a superior tool will make this the gold standard for surgeries that center around bone/cartilage harvesting and facial reconstruction. Surgeons can use this tool to improve their patient's satisfaction outcomes, and patients will be drawn to surgeons that use this tool because of the better postoperative outcomes it achieves. This is highly prevalent among plastic surgery, including craniofacial surgeons and otolaryngologists.

From a hospital standpoint, an improvement in satisfaction rates among patients and families will result in higher reimbursements that are based on outcomes and quality improvement surveys. Additionally, a decrease in revision surgeries will reduce hospital expenditure on preventable surgeries. Functionally, having a tool that provides more precise cuts and control will improve the functional and physiological outcomes in patients that are dependent on the quality of bone/cartilage grafts. This is especially true in orthopedic surgery and neurosurgery.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention.

The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A handle assembly for a gouge, comprising:
a handle, wherein a distal end of the handle includes first threads;
a grip that has an interior channel, wherein at least a portion of the interior channel includes second threads that mate with the first threads on the distal end of the handle to secure the grip to the handle;
a collet, wherein a distal end of the collet includes an opening that is sized to receive a proximal end of a gouge; and
a collar that is sized to receive the collet, wherein the collar mounts onto a distal end of the grip.

2. The handle assembly of claim 1, wherein the grip includes a distal portion that has third threads, wherein the collar includes fourth threads internal to the collar, and wherein the fourth threads mate with the third threads to secure the collar to the grip.

3. The handle assembly of claim 2, wherein the collet includes a pair of slots that extend to the opening in the collet, and wherein the pair of slots compress the opening in response to the fourth threads mating with the third threads to secure the collar to the grip.

4. The handle assembly of claim 3, wherein the pair of slots extend from the opening in the collet to a central portion of the collet.

5. The handle assembly of claim 3, wherein the pair of slots is configured to receive at least a portion of the gouge.

6. The handle assembly of claim 3, wherein compression of the opening secures the gouge within the opening at a desired cutting depth and a desired angle.

7. The handle assembly of claim 1, wherein the grip includes a sleeve positioned in between the distal end of the grip and a proximal end of the grip.

8. The handle assembly of claim 1, wherein the interior channel of the grip runs an entire length of the grip.

9. The handle assembly of claim 1, wherein the interior channel includes a distal channel portion and a proximal channel portion, and wherein the distal channel portion tapers down in size to connect to the proximal channel portion.

10. The handle assembly of claim 1, wherein the collar includes an opening that aligns with the opening in the collet when the collet is positioned within the collar.

11. A method of forming components of a handle assembly for a gouge, the method comprising:
forming a handle with a proximal end and a distal end, wherein the distal end of the handle is formed to include first threads;
forming a grip that has an interior channel, wherein at least a portion of the interior channel includes second threads that mate with the first threads on the distal end of the handle to secure the grip to the handle;
forming a collet such that a distal end of the collet includes an opening that is sized to receive a proximal end of a gouge; and
forming a collar that is sized to receive the collet, wherein the collar mounts onto a distal end of the grip.

12. The method of claim 11, further comprising:
mounting the grip to the handle by mating the first threads with the second threads;
positioning the collet within the collar; and
mounting the collar to the distal end of the grip.

13. The method of claim 11, further comprising forming the grip to include a distal portion that has third threads, wherein the collar is formed to include fourth threads internal to the collar, and wherein the fourth threads mate with the third threads to secure the collar to the grip.

14. The method of claim 13, further comprising forming the collet to include a pair of slots that extend to the opening in the collet, and wherein the pair of slots compress the opening in response to the fourth threads mating with the third threads to secure the collar to the grip.

15. The method of claim 14, wherein the pair of slots extend from the opening in the collet to a central portion of the collet.

16. The method of claim 14, wherein the collet is formed to include two pair of slots that extend to the opening in the collet.

17. The method of claim 14, wherein the pair of slots is configured to receive at least a portion of the gouge.

18. The method of claim 11, wherein the interior channel of the grip runs an entire length of the grip.

19. The method of claim 18, wherein the interior channel includes a distal channel portion and a proximal channel portion, and wherein the distal channel portion tapers down in size to connect to the proximal channel portion.

20. The method of claim 11, wherein the collar is formed to include an opening that aligns with the opening in the collet when the collet is positioned within the collar.
